(19) United States
(12) Patent Application Publication
(10) Pub. No.: US 2002/0185331 A1
(43) Pub. Date: Dec. 12, 2002

(54) Tunable stethoscope head assembly

(76) Inventors: Richard J. Deslauriers, Woodbury, CT (US); Lewis W. Chappel, New Hartford, CT (US); Robert T. Potash, South Windsor, CT (US)

Correspondence Address: RICHARD J. DESLAURIERS, 143 WOLCOTT ROAD, WOLCOTT, CT, 06716, US

(21) Appl. No.: 09/878,217

(22) Filed: Jun. 12, 2001

(51) Int. Cl. A61B 7/02

(52) U.S. Cl. 181/131; 181/130

(57) ABSTRACT

This application discloses a stethoscope that utilizes a dual diaphragm construction such that the user of the stethoscope is able to tune in to or to allow the user to select between the isolation and accentuation of either high frequency sounds or low frequency sounds while using a single stethoscope and without the removal of said stethoscope from the patient's skin.

### CROSS-REFERENCES TO RELATED APPLICATIONS

Not Applicable

### STATEMENT REGARDING FEDERALLY SPONSORED RESEARCH OR DEVELOPMENT

Not Applicable

### REFERENCE TO A “MICROFICHE APPENDIX”

Not Applicable

### BACKGROUND OF THE INVENTION

1. Field of the Invention

This invention relates to the field of medical diagnostic instruments. In particular this invention relates to the field of auscultation, which entails the use of stethoscopes in the diagnosis of patients by medical professionals. Acoustic stethoscopes are one type of stethoscope in the field of medical diagnosis that may be used for the capture of either low or high frequency heart and lung sounds in a patient.

2. Description of the Related Art

The prior art discloses a number of different kinds of stethoscopes and variations thereof. Conventional acoustic stethoscopes are disclosed by the prior art that function through sound vibrations colliding with the diaphragm of the stethoscope, and whereas the vibrating or resonating of the diaphragm then causes the reproduction of sound which travels through the acoustic cavity of the stethoscope and to the user's ears through the stethoscope's tubing.

Stethoscopes manufactured with multiple heads that allow a user to accentuate multiple frequency ranges with the same device are well known in the field. One side of such stethoscope is the diaphragm side that is intended for picking up high frequency sounds. Another side may be a bell side that is attuned to reproducing low frequency sounds. Although these multiple sided stethoscopes facilitate a user's ability to isolate both high and low frequency sounds within the body, the user is required to remove the stethoscope from the patient's body and then reapply a different side of the stethoscope. This process of switching sides takes time and makes it difficult to find the precise area of the patient's body that the user had previously placed the stethoscope, and thereby adversely affect the user's ability to make a quick and accurate diagnosis.

This invention seeks to introduce a multiple frequency response stethoscope head, which allows a user to accentuate and reproduce low frequency sounds to high frequency sounds without removing the stethoscope head from the patient's body through utilizing a dual diaphragm stethoscope head construction, which process of changing the frequency response is characterized as “Tuning”.

### BRIEF SUMMARY OF THE INVENTION

The preferred embodiment discloses a tunable stethoscope that comprises a single sided stethoscope head whereby the stethoscope can be tuned to different frequencies enabling the pick up of both low frequency sound vibrations and high frequency sound vibrations. The manufacture of stethoscope heads is well disclosed in the prior art and any number of techniques can be used to manufacture the shell of a single sided stethoscope head.

Pursuant to the preferred embodiments, this application discloses a stethoscope head that permits a user to isolate and reproduce both high and low frequency sound resonating from a patient without removing the stethoscope's head from its position on the patient's skin. The preferred embodiment allows the user to select different Tunings for the diaphragm by varying the degree of pressure the user applies to the stethoscope head. According to the preferred embodiment of the present invention, applying the stethoscope head, herein disclosed, to a patient's skin with minimal contact pressure isolates low frequency resonations, while the stethoscope head remains unresponsive to high frequency resonations. Further, through increased contact pressure between the stethoscope head and the patient's skin, high frequency sounds are isolated, whereby the disclosed stethoscope remains unresponsive to impact with low frequency resonations.

The preferred embodiment of this invention discloses a single sided stethoscope head assembly that utilizes two diaphragms of different densities and flexibilities, whereas the combination of the two preceding factors result in the acoustic stiffness of the diaphragm determining the diaphragms frequency response range. The outer diaphragm of this invention is elastomeric with minimal acoustic stiffness exhibiting increased responsiveness and flexibility for accentuating low frequency pickup. The inner diaphragm is denser and semi-rigid in its acoustic stiffness thereby facilitating the pickup of high frequency sounds. The user chooses between the two diaphragms by applying minimal contact pressure between this stethoscope head and the patient's skin to activate only the exterior diaphragm and through increased contact pressure the user activates the inner diaphragm.

According to the preferred embodiment of the present invention, the inner diaphragm is such that sound waves pass unobstructed through one or more holes located in the surface of the inner diaphragm, such that the inner diaphragm does not resonate in the disclosed invention's low frequency mode. Thus, the preferred embodiment allows the low frequency sound waves picked up by the outer diaphragm, in its relaxed state to pass unobstructed through the one or more air holes in the inner diaphragm and travel through to stethoscope's headset without impacting the high frequency response diaphragm.

According to another preferred embodiment, the outer elastomeric diaphragm is treated with or comprised of antibiotics or anti-microbial compound in order to promote sterility in the use of the disclosed invention from subject to subject.

According to another preferred embodiment of the present invention, the outer elastomeric diaphragm can be easily discarded and replaced with a new outer diaphragm, which further promotes the sterility of the disclosed invention through prolonged use of this invention.

Further the disclosed tunable stethoscope head is universally adaptable to any stethoscope lumen, such that the disclosed invention can be mated to either a single lumen or multiple lumen stethoscope tube.

According to another embodiment of the present invention, a tunable stethoscope head is incorporated in a standard dual sided acoustic stethoscope with different size heads, which thereby makes this invention suitable for use with both an adult or a pediatric patient.

### BRIEF DESCRIPTION OF THE SEVERAL VIEWS

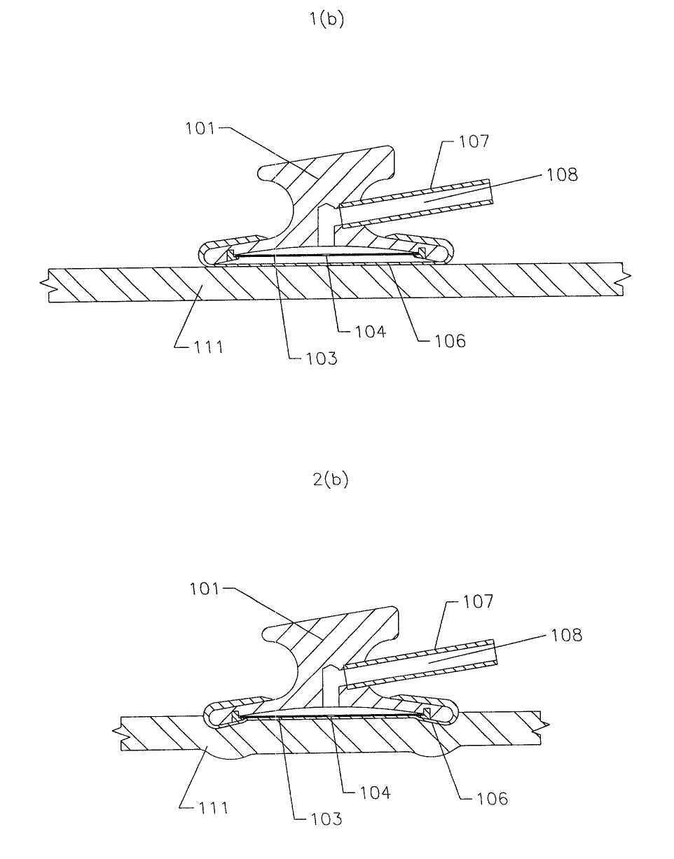

FIG. 1(a) shows a side view schematic of the tunable stethoscope head in its relaxed state (Low Frequency Mode).

FIG. 1(b) shows a side view schematic of the tunable diaphragm in its flexed state (High Frequency Mode).

FIG. 2(a) depicts a side view schematic of the tunable stethoscope in its relaxed state as applied to a subject (Low Frequency Mode).

FIG. 2(b) depicts a side view schematic drawing of the tunable stethoscope in its flexed state being applied to a subject (Low Frequency Mode).

### DETAILED DESCRIPTION OF THE INVENTION

In referring to the followings drawings, whereby like reference numbers refer to like parts throughout the various views.

FIGS. 11(a) and 11(b) show side-view schematics of this tunable stethoscope head, which depict the preferred embodiments of the present invention. The disclosed invention functions through the incorporation of two separate diaphragms, Hard Diaphragm (“HD”) 103 and Elastomeric Diaphragm (“ED”) 106, having both diaphragms incorporated into a singular housing of a stethoscope head, Shell 101. Shell 101 has a concave receiving portion that houses the HD 103 and ED 106, wherefrom the internal mechanics of the disclosed tunable stethoscope permit Tuning to multiple frequency ranges from a single subject.

Shell 101 has top section, referred to herein as Plate 109, and which is used as the focal point whereat the user applies pressure to hold the stethoscope against the subject. By the user applying minimal pressure to Plate 109 the user selects the low frequency response mode, or wherefrom the increased application pressure to Plate 109 until ED106 contacts HD 103 the disclosed invention tunes to high frequency resonations.

ED 106 is the outer diaphragm, whereby ED 106 is comprised of a chemically inert and durable elastomer, such as Kraton or C-Flex™, and whereas ED 106 is particularly responsive to low frequency sound waves falling between 0 and 250 Hz. ED 106 is approximately parabolic in cross-section with a curled peripheral edge such as to create a lip, whereas the lip forms an opening in ED 106 with a diameter smaller than the diameter of Shell 101, and which lip is then used as a method of attachment around the exterior rim of Shell 101. HD 103 is made of a semi-rigid material that allows only for minimal flex that is useful in connection with accentuating high frequency sound modulations over 200 Hz.

HD 103 is inserted into the open end of Shell 101, whereby the open end is manufactured as to allow for clearance 105. HD 103 is held in Clearance 105 through the use of Support Ring 102, and which support Ring 102 is sized as to have a diameter minimally larger than HD 103 with a minimal width so that Support Ring 102 merely prevents HD 103 from falling out of Shell 101 without affecting HD 103's flexibility. Support Ring 102 mates with Groove 110, which is manufactured into the inner surface of the open end of Shell 101, such that Support Ring 102 fits securely within Groove 110 and locks into place.

HD 103 has Air Hole 104, a circular hole no greater than 1″ in diameter, beneath the opening of Sound Path 108, and whereby low frequency sound waves generated by ED 106 pass through Air Hole 104, and thereby, Air Hole 104 prevents their impact with HD 103 preventing HD 103's high frequency response. Sound Path 108 is an unobstructed pathway, bored through Shell 101, which carries sound energy created by the diaphragms to Fixed Barb 107. Fixed Barb 107 is a hollow elongated member which is used as a means of attaching Shell 101 to tubing of the stethoscope, and whereby said attachment of the tubing to Fixed Barb 107 is accomplished in any suitable manner, such as glue or adhesive, whether known or not yet discovered in the field. Fixed Barb 107 is attached to Shell 101 by physical insertion into Sound Path 108 in Shell 101, whereas such methods of insertion and attachment are well known in the field.

FIG. 1(b) shows the tunable stethoscope head if operating under its high frequency mode. ED 106 flexes to the point of contact with inner HD 103, and whereat said point of contact ED 106 and HD 103 act in concert as one diaphragm and further, the joint action increases the acoustic stiffness and enables the accentuation of high frequency sounds in the diaphragms as herein described. Sound created by the vibrations of one or both of the diaphragms is carried through Sound Path 108, which is bored through Shell 101 and on through Fixed Barb 107.

FIGS. 2(a) and 2(b) are depictions of the preferred embodiments of the present invention while being applied to a contact surface, such as the skin of a patient, and being further characterized as the Subject Wall 111. Pursuant to this invention, when the tunable stethoscope is applied to Subject Wall 111, resistance is applied to ED 106, whereby resistance causes ED 106 to flex. When minimal pressure is applied, as shown in FIG. 2(a), ED 106 does not flex to the point of contact with inner HD 103. When the disclosed invention is applied to Subject Wall 111, such as to listen to internal human body sounds through its application to the subject's skin, low frequency modulations within are picked up by the flexible outer ED 106. ED 106 responds by transmitting those low frequency modulations to the user through Air Hole 104 in HD 103, without impacting and activating HD 103 and, thus, HD 103 remains illusory in this stethoscope's low frequency mode.

FIG. 2(b) shows the disclosed invention being tuned to high frequency sound modulations accomplished through the user increasing the pressure being applied to Plate 109 resulting in additional force being applied to Subject Wall 111. As a consequence of increasing the pressure, ED 106 and HD 103 are forced together to the point of their contact effectively closing off Air Hole 104, and therefore when sound waves impact the outer ED 106 the sound waves are impacting HD 103 at the same time. Thus, whereas ED 106 and HD 103 are acting in concert with increased acoustic stiffness, the now operative HD 103 enable the disclosed invention to tune into high frequency resonations.

What is claimed:

1. A dual diaphragm tunable stethoscope head comprised of a machined or molded solid form, which form is comprised of plastics or metals, forming a body or shell having a first recessed section, whereas the section regresses from an exterior rim parabolicly to the center of the body, whereat a bored sound passage extends unobstructed from such central portion through the second portion of the shell, which exterior portion of said second section is formed as a handle to hold the stethoscope head, whereto an elongated member, the stethoscope barb, is used as a means of connecting the body of the stethoscope to standard single lumen or a dual lumen stethoscope tubing and which stethoscope tubing is connected to the stethoscope headset, such that
a, first, inner diaphragm that is made of a semi-rigid plastic, or similar material that is rigid in its acoustic stiffness, being responsive to high frequency sound resonations greater than or equal to 200 Hz, and whereat the center of said inner diaphragm has one or more air holes placed in said inner diaphragm; and
a, second, outer diaphragm that is a molded elastomer that covers the entire open end of the stethoscope shell, and whereas said elastomer is flexible and its acoustic stiffness being responsive to impact from low frequency sound waves between 0 and 250 Hz.

2. The stethoscope head designed in accordance with claim 1, such that this stethoscope functions through the utilization of a high frequency and a low frequency response diaphragm both being enclosed in a single stethoscope shell, whereas
the outer elastomeric diaphragm is used as the sole diaphragm in this invention's low frequency mode, whereas, through minimal pressure between the stethoscope head and the patient's skin reproduces low frequency sound, whereas sound waves impact with the low frequency elastomeric diaphragm resulting in low frequency sound waves, passing through the air hole(s) in hard diaphragm so as not to cause vibration of the hard diaphragm, and continuing to travel through the sound path and into the stethoscope barb; and whereby
when operating in the disclosed invention's high frequency mode, pressure is applied to the head of the stethoscope until the point whereat the outer elastomeric diaphragm contacts the inner semi-rigid diaphragm which effectively closes off the air hole(s) in the inner diaphragm, and which causes the two diaphragms to act in concert as one diaphragm being of an increased acoustic stiffness that is effective in its reproduction and transmission of high frequency sound waves.

3. The stethoscope head design in accordance with claim 1 whereas the outer elastomeric diaphragm is treated or manufactured with anti-microbial compounds that actively promote sterility in the contact portion of this stethoscope.

4. The stethoscope head design in accordance with claim 3 whereas the outer elastomeric diaphragm is easily attached and removed for simplified replacement of the contact portion of the stethoscope to promote the disclosed invention's sterility.

5. The stethoscope head design in accordance with claim 3 whereas the outer elastomeric diaphragm is permanently secured to the shell of the disclosed invention.

6. The stethoscope head design in accordance with claim 3 whereas such dual diaphragm is incorporated into any two-sided stethoscope.

7. The stethoscope head designed in accordance with claim 6, whereas such two-sided stethoscope has one stethoscope sized for use with adults and the other side is sized for use with pediatric or infant patients.
